(19) United States
(12) Patent Application Publication
(10) Pub. No.: US 2018/0070902 A1
(43) Pub. Date: Mar. 15, 2018

(54) APPARATUS AND METHOD FOR 4D X-RAY IMAGING

(71) Applicant: Carestream Health, Inc., Rochester, NY (US)

(72) Inventors: Yuan Lin, Rochester, NY (US); William J. Sehnert, Fairport, NY (US)

(21) Appl. No.: 15/611,900

(22) Filed: Jun. 2, 2017

Related U.S. Application Data
(60) Provisional application No. 62/394,232, filed on Sep. 14, 2016.

(51) Int. Cl. A61B 6/00; A61B 5/107; A61B 6/03; A61B 5/00; G06T 11/00

(52) U.S. Cl. CPC A61B 6/486; A61B 6/54; A61B 5/1077; A61B 5/1079; A61B 6/4417; A61B 6/4085; G06T 2210/41; A61B 6/466; A61B 6/5205; A61B 6/5247; A61B 5/0035; A61B 5/0077; G06T 11/006; A61B 6/03

(57) ABSTRACT

A system for reconstructing a 4D image has a surface acquisition system for generating a 3D surface model of an object and an X-ray imaging system for acquiring at least one 2D X-ray projection image of the object. A controller controls the surface acquisition system and the X-ray imaging system. A processor applies a 4D reconstruction algorithm/method to the 3D surface model and the at least one 2D X-ray projection to reconstruct a 4D X-ray volume of the imaged body part in motion.

### CROSS REFERENCE TO RELATED APPLICATIONS

This application claims the benefit of U.S. Provisional Application No. 62/394,232, filed Sep. 14, 2016, entitled APPARATUS AND METHOD FOR 4D X-RAY IMAGING by Lin et al., which is hereby incorporated by reference in its entirety.

### TECHNICAL FIELD

The present disclosure relates, in general, to the field medical imaging (such as, fluoroscopy, computed tomography [CT], tomosynthesis, low-cost CT, magnetic resonance imaging [MRI], and PET, and the like. In particular, the disclosure presents an apparatus and a method for reconstructing motion 3D objects, which is considered to be four-dimensional (4D) imaging.

### BACKGROUND OF THE INVENTION

An X-ray imaging scanner is useful to diagnose some joint disorders, particularly a 4D X-ray imaging scanner.

For example, refer to the introduction section of the following reference which provides background information: Yoon Seong Choi, et al., “Four-dimensional real-time cine images of wrist joint kinematics using dual source CT with minimal time increment scanning”. Yonsei Medical Journal, 2013. 54(4): p. 1026-1032.

One paragraph of the Choi reference states: “In the past, radiologic studies of joint disorders focused mainly on the static morphologic depiction of joint internal derangements. However, some joint disorders may not show definite abnormalities in a static radiologic study, but will still have dormant abnormalities that are aggravated with joint movement, which triggers the need for radiologic imaging of dynamic joint movement. The wrist joint in particular requires four-dimensional (4D) dynamic joint imaging because the wrist is an exceedingly complex and versatile structure, consisting of a radius, ulna, eight carpals, and five metacarpals all engaged with each other. Each of these carpal bones exhibits multiplanar motion involving significant out-of-plane rotation of bone rows, which is prominent during radio-ulnar deviation. The kinematics of these carpal bones have been not fully elucidated. Thus, studies using 4D wrist imaging were conducted to determine the proper modality and to investigate carpal kinematics.”

Current techniques to obtain 4D images of a moving joint mainly rely on utilizing a multi-detector CT (MDCT) (such as described in the above-mentioned reference). This current technique is considered by some practitioners to have at least two disadvantages. First, it needs a high-end MDCT scanner (e.g., the mentioned reference used a dual source CT scanner, SOMATOM Definition Flash, manufactured by Siemens Medical, Forchheim, Germany). This high-end MDCT has multiple X-ray tubes and fast rotation speed, which can help reconstruct dynamic images with fine temporal resolution. Second, this technique is viewed as inducing excessive radiation dose, and can potentially cause cancer to the patients.

In view of these disadvantages, this disclosure proposes a system and method to reconstruct 4D images.

The discussion above is merely provided for general background information and is not intended to be used as an aid in determining the scope of the claimed subject matter.

### SUMMARY

Certain embodiments described herein address the need for methods that generate 4D images for diagnostic imaging. Methods of the present disclosure combine aspects of 3D volume imaging from computed tomography (CT) apparatus that employs radiographic imaging methods with surface imaging capabilities provided using structured light imaging or other visible light imaging method.

These aspects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to an embodiment of the present disclosure, there is provided a system for reconstructing a 4D image, comprising: a surface acquisition system for generating a 3D surface model of an object; an X-ray imaging system for acquiring at least one 2D X-ray projection image of the object; a controller to control the surface acquisition system and the X-ray imaging system; and a processor to apply a 4D reconstruction algorithm/method to the 3D surface model and the at least one 2D X-ray projection to reconstruct a 4D X-ray volume of the imaged body part in motion.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIGS. 1A through 1D illustrate a dynamic imaging apparatus using a surface acquisition system employed to capture/record/obtain a motion 3D surface model of the body part of interest.

FIGS. 1E through 1H illustrate an X-ray imaging system employed to acquire a series of 2D projection images of the body part.

FIG. 2A is a schematic view that shows components of a CBCT image capture and reconstruction system.

FIG. 2B is a schematic diagram that shows principles and components used for surface contour acquisition using structured light.

FIG. 3A is a top view schematic diagram of a CBCT imaging apparatus using a rotational gantry for simultaneously acquiring surface contour data using a surface contour acquisition device during projection data acquisition with an X-ray tube and detector.

FIG. 3B is a top view schematic diagram of a CBCT imaging apparatus using a rotational gantry for simultaneously acquiring surface contour data using multiple surface contour acquisition devices during projection data acquisition with an X-ray tube and detector.

FIG. 3C is a top view schematic diagram of an imaging apparatus for a multi-detector CT (MDCT) system using one surface contour acquisition device affixed to the bore of the MDCT system during projection data acquisition.

FIG. 3D is a schematic top view showing an imaging apparatus for chest tomosynthesis using multiple surface contour acquisition devices placed outside of the imaging system.

FIG. 3E is a schematic top view diagram that shows a computed tomography (CT) imaging apparatus with a rotating subject on a support and with a stationary X-ray source X-ray detector and multiple surface contour acquisition devices.

FIG. 3F is a schematic view diagram that shows an extremity X-ray imaging apparatus with multiple surface acquisition devices that can move independently on rails during projection data acquisition.

FIG. 3G is a schematic top view showing an imaging apparatus for chest radiographic imaging using multiple surface contour acquisition devices positioned outside of the imaging system.

FIG. 4 is a schematic diagram that shows change in voxel position due to patient movement.

FIG. 5 is a logic flow diagram illustrating a method using the analytical form reconstruction algorithm for 3D motion reduction.

FIG. 6 is a logic flow diagram illustrating a method using the iterative form reconstruction algorithm for 3D motion reduction.

FIG. 7A shows a computed tomography image illustrating blurring and double images caused by motion.

FIG. 7B shows a computed tomography image illustrating long range streaks caused by motion.

FIG. 8 shows a respiratory motion artifact for a chest scan.

FIGS. 9A and 9B show positions of a hand bending or flexion during a volume imaging exam.

FIG. 9C shows an angular distance between beginning and ending positions shown in FIGS. 9A and 9B.

FIG. 10 is a logic flow diagram showing a sequence for generating 4D image content according to an embodiment of the present disclosure.

FIG. 11A is a schematic diagram that shows basic volume transformation for a reconstructed volume according to 3D surface contour characterization.

FIG. 11B is a schematic diagram that shows how the 3D transformation is applied to the skeletal features and other inner structure of the imaged subject.

FIG. 11C is a schematic diagram showing application of the 3D transformation in enlarged form.

FIG. 11D is a schematic showing obtaining an acquired radiographic projection.

FIG. 11E is a schematic diagram that shows calculating a forward projection corresponding to the acquired radiographic projection of FIG. 11D.

### DETAILED DESCRIPTION OF THE DISCLOSURE

The following is a detailed description of the embodiments of the invention, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

As used herein, the term “energizable” relates to a device or set of components that perform an indicated function upon receiving power and, optionally, upon receiving an enabling signal.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the term “subject” is used to describe the object that is imaged, such as the “subject patient”, for example.

Radio-opaque materials provide sufficient absorption of X-ray energy so that the materials are distinctly perceptible within the acquired image content. Radio-translucent or transparent materials are imperceptible or only very slightly perceptible in the acquired radiographic image content.

In the context of the present disclosure, “volume image content” describes the reconstructed image data for an imaged subject, generally stored as a set of voxels. Image display utilities use the volume image content in order to display features within the volume, selecting specific voxels that represent the volume content for rendering a particular slice or view of the imaged subject. Thus, volume image content is the body of resource information that is obtained from a radiographic or other volume imaging apparatus such as a CT, CBCT, MDCT, MRI, PET, tomosynthesis, or other volume imaging device that uses a reconstruction process and that can be used to generate depth visualizations of the imaged subject.

Examples given herein that may relate to particular anatomy or imaging modality are considered to be illustrative and non-limiting. Embodiments of the present disclosure can be applied for both 2D radiographic imaging modalities, such as radiography, fluoroscopy, or mammography, for example, and 3D imaging modalities, such as CT, MDCT, CBCT, tomosynthesis, dual energy CT, or spectral CT.

In the context of the present disclosure, the term “volume image” is synonymous with the terms “3 dimensional image” or “3D image”.

In the context of the present disclosure, a radiographic projection image, more simply termed a “projection image” or “x-ray image”, is a 2D image formed from the projection of x-rays through a subject. In conventional radiography, a single projection image of a subject can be obtained and analyzed. In volume imaging such as CT, MDCT, and CBCT imaging, multiple projection images are obtained in series, then processed to combine information from different perspectives in order to form image voxels.

Embodiments of the present disclosure are directed to apparatus and methods that can be particularly useful with volume imaging apparatus such as a CBCT system.

A description of a suitable 4D X-ray imaging scanner is described below. Generally, a 4D X-ray imaging scanner is comprised of two systems: (i) a surface acquisition system and (ii) an X-ray imaging system. In a preferred arrangement, the two systems are calibrated to one coordinate system and are synchronized.

Applicants now describe the imaging process employing a 4D X-ray imaging scanner.

When Applicants' system is employed for medical purposes, the object being imaged is anatomy (body part). For ease of illustration/presentation of the system, an anatomy of a hand is described.

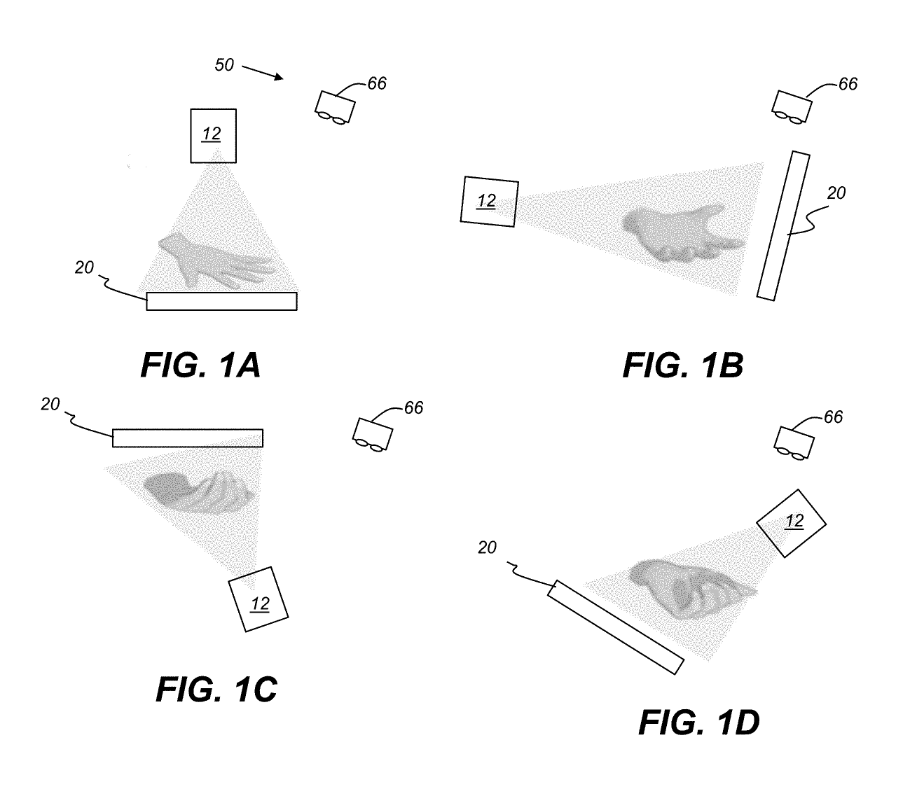

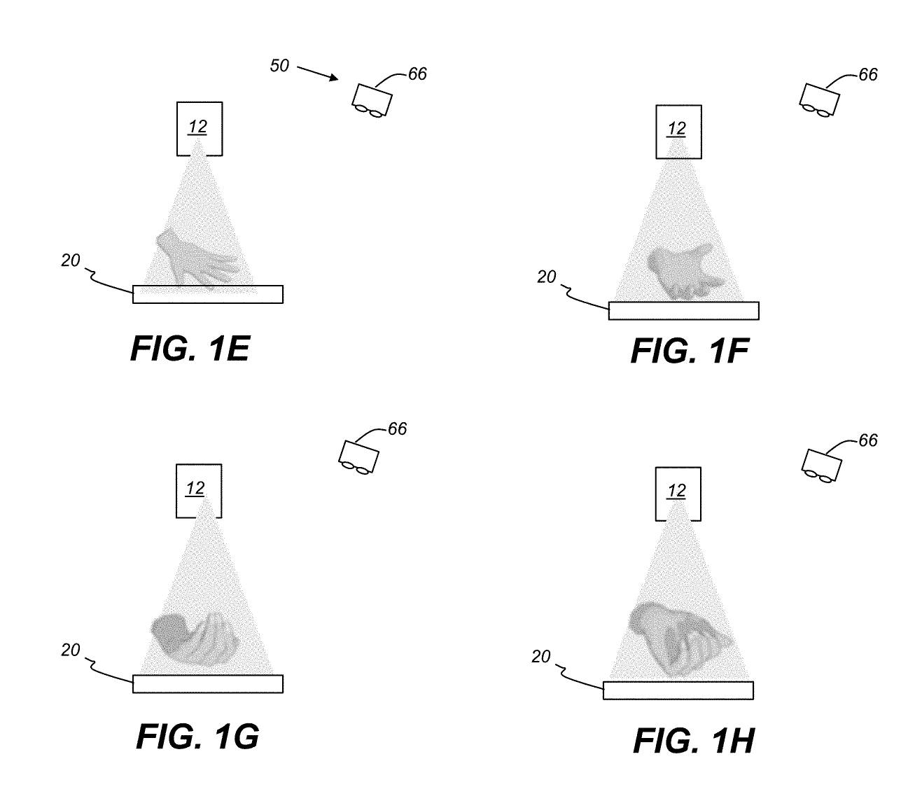

In a FIRST STEP, a surface acquisition system 50 that includes a camera 66 is employed to capture/record/obtain a motion 3D surface model of the body part in the field of view. Refer to FIGS. 1A through 1D, wherein the hand is presented in various positions.

In a SECOND STEP, the X-ray imaging system that includes a source 12 and a detector 20 acquires a series of 2D projection images of the body part. Refer to FIGS. 1E through 1H, wherein the hand is presented in various positions.

Various modalities can be employed for the surface acquisition system and the X-ray imaging system, for example: CT, fluoroscopy, tomosynthesis, and radiography. In a preferred arrangement, the surface acquisition system and the X-ray imaging system are different modalities.

Applicants note that the geometry for the system's X-ray tube and X-ray detector can be either stationary like a radiography/fluoroscopy system (as illustrated in FIGS. 1E-1H) or dynamic like a CT/tomosynthesis system (as illustrated in FIGS. 1A-1D).

In a THIRD STEP, after image acquisition, some (or all) of the acquired images are employed to reconstruct the surface of the hand/object. Techniques are known to reconstruct the surface of a moving object. Two examples are referenced, and incorporated herein in their entirety:

(1) Sinha, Ayan, Chiho Choi, and Karthik Ramani. “Deep Hand: Robust Hand Pose Estimation by Completing a Matrix Imputed With Deep Features.” Proceedings of the IEEE Conference on Computer Vision and Pattern Recognition (CVPR) 2016, pp. 4150-4158 (with video content available at the https://www. address “youtube.com/watch?v=ScXCgC2SNNQ&ab_channel=CdesignLab”.); and

(2) Huang, Chun-Hao, et al. “Volumetric 3D Tracking by Detection.” 2016 IEEE Conference on Computer Vision and Pattern Recognition (CVPR), 2016, pp. 3862-3870 (with video content available at the “https://www. address youtube.com/watch?v=zVavXrcyeYg&ab_channel=Chun-HaoHuang”.)

In a FOURTH STEP, after image acquisition, some (or all) of the acquired images are employed to reconstruct at least one 4D image. This is accomplished by the following sequence of steps, for each 2D X-ray projection image: a) the reconstructed volume is deformed according to the captured motional 3D surface model; b) the reconstructed volume is adjusted according to patient anatomical structures or implants, such that the forward projection of the reconstructed volume can match the acquired 2D X-ray projection image; and c) a reconstruction algorithm (e.g., FBP (filtered back projection) or iterative reconstruction algorithms) is performed/applied to update the volume.

In a FIFTH STEP, after reconstruction, one or more of the 4D images can be displayed, stored, or transmitted.

While the making and use of various embodiments are described, it should be appreciated that the specific embodiments described herein are merely illustrative of specific ways to make and use the system and do not limit the scope of the invention.

Applicants have described a system for reconstructing a 4D image. The system includes: (a) a surface acquisition system for generating a 3D surface model of an object; (b) an X-ray imaging system for acquiring at least one 2D X-ray projection image of the object; (c) a controller to control the surface acquisition system and the X-ray imaging system; and (d) a processor to apply a 4D reconstruction algorithm/method to the 3D surface model and the at least one 2D X-ray projection to reconstruct 4D X-ray volume of the imaged body part in motion.

In one arrangement, the surface acquisition system comprises: (a) one or more light sources adapted to project a known pattern of light grid onto the object using one or more light sources; (b) one or more optical sensors adapted to capture a plurality of 2D digital images of the object; and (c) a surface reconstruction algorithm for reconstructing the 3D surface model of the object using the at least one 2D projection image.

In one arrangement, the light sources and the optical sensors are adapted to be either (i) mounted to a rotational gantry of the X-ray imaging system, (ii) affixed to the bore of the X-ray imaging system, or (iii) placed outside of (separate from) the X-ray imaging system.

In one arrangement, the X-ray imaging system comprises: (a) one or more X-ray sources adapted to controllably emit X-rays; and (b) one or more X-ray detectors including a plurality (optionally: of rows) of X-ray sensors adapted to detect X- rays that are emitted from the X-ray sources and have traversed the object.

In one arrangement, the X-ray sources and X-ray detectors move in a trajectory, wherein the trajectory includes, but is not limited to, a helix (e.g., MDCT), full circle (e.g., dental CBCT CT), incomplete circle (e.g., extremity CBCT), line, sinusoid, and stationary (e.g., low-cost CT), and the like.

In one arrangement, the controller synchronizes the surface imaging system and the X-ray imaging system.

In one arrangement, the 4D reconstruction algorithm/method comprises: (a) a X-ray projection correction process/method/algorithm to generate a corrected 2D X-ray projection; (b) a 3D surface deformation algorithm/method/process to deform each 3D surface model to the next time-adjacent 3D surface model and generate at least one transformation parameter; (c) a 3D volume deformation algorithm/method/process to deform the volume under reconstruction according to the at least one transformation parameter; (d) a 3D volume deformation algorithm/method/process to deform the volume under reconstruction according to the 2D X-ray projection using an anatomical structure or implant; and (e) an analytical form reconstruction algorithm/method/process or an iterative form reconstruction algorithm/method/process.

In one arrangement, the X-ray projection correction process/method/algorithm includes (but is not limited to) a scatter correction, a beam hardening correction, a lag correction, a veiling glare correction, or a metal artifact reduction correction.

In one arrangement, the system further comprises a 3D surface registration algorithm comprising a rigid-object registration algorithm or a deformable registration algorithm.

In one arrangement, the analytical form reconstruction algorithm/method/process includes an FDK (Feldkamp-Davis-Kress) algorithm.

In one arrangement, the iterative form reconstruction algorithm/method/process includes a SART algorithm, a statistical reconstruction algorithm, a total variation reconstruction algorithm, or an iterative FDK algorithm.

In one arrangement, the 4D reconstruction method is applied until a predetermined threshold criterion is met (e.g., for example, a predetermined number of iterations or a maximum error less than a threshold error value).

### Detailed Information Regarding a 4D X-ray Imaging Scanner

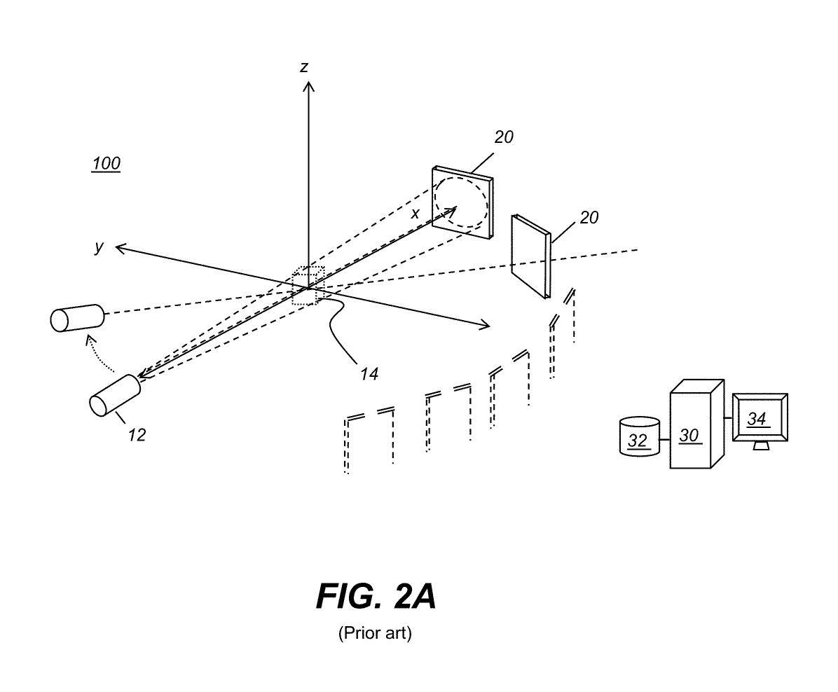

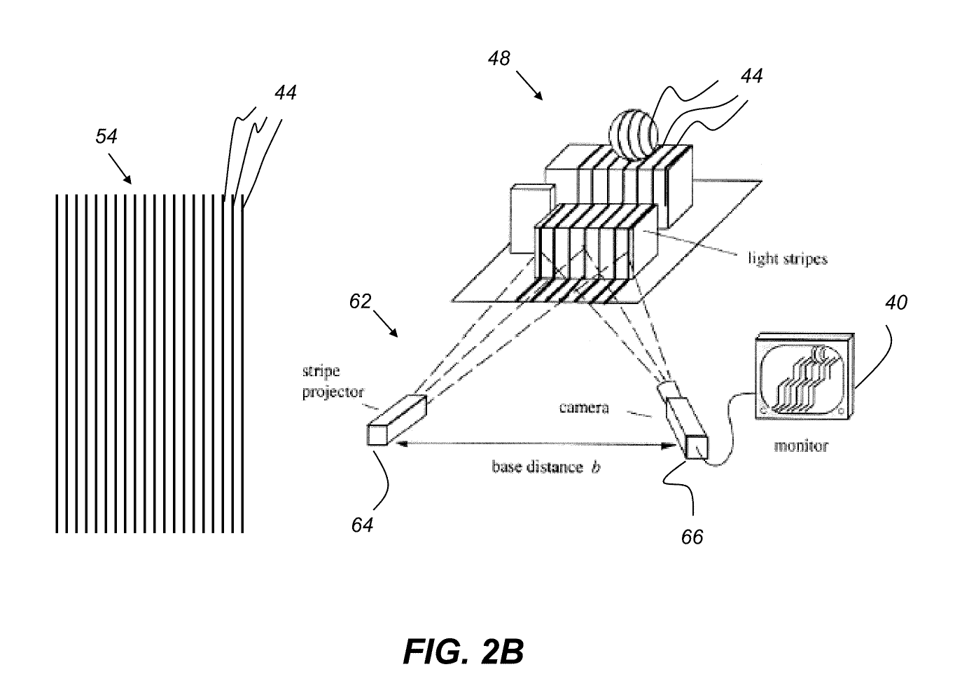

To more particularly understand the methods of the present disclosure and the problems addressed, it is instructive to review principles and terminology used for 3D volume image capture and reconstruction. Referring to the perspective view of FIG. 2A, there is shown, in schematic form and using enlarged distances for clarity of description, the activity of a conventional CBCT imaging apparatus 100 for obtaining, from a sequence of 2D radiographic projection images, 2D projection data that are used to reconstruct a 3D volume image of an object or volume of interest, also termed a subject 14 in the context of the present disclosure. Cone-beam radiation source 12 directs a cone of radiation toward subject 14, such as a patient or other subject.

For a 3D or volume imaging system, the field of view (FOV) of the imaging apparatus is the subject volume that is defined by the portion of the radiation cone or field that impinges on a detector for each projection image. A sequence of projection images of the field of view is obtained in rapid succession at varying angles about the subject, such as one image at each 1-degree angle increment in a 200-degree orbit. X-ray digital radiation (DR) detector 20 is moved to different imaging positions about subject 14 in concert with corresponding movement of radiation source 12.

FIG. 2A shows a representative sampling of DR detector 20 positions to illustrate schematically how projection data are obtained relative to the position of subject 14. Once the needed 2D projection images are captured in this sequence, a suitable imaging algorithm, such as filtered back projection (FBP) or other conventional technique, is used for reconstructing the 3D volume image. Image acquisition and program execution are performed by a computer 30 or by a networked group of computers 30 that are in image data communication with DR detector 20. Image processing and storage is performed using a computer-accessible memory 32. The 3D volume image can be presented on a display 34.

### Surface Contour Acquisition

In order to track patient motion during projection image acquisition, the imaging apparatus needs sufficient data for detecting surface displacement. To obtain this surface modeling information, an embodiment of the present disclosure can employ surface contour acquisition, such as contour acquisition using structured light imaging.

FIG. 2B shows surface contour acquisition principles, in schematic form. Surface contour acquisition can be provided from a scanner 62 having a projector 64 that directs a pattern 54 (for example, a pattern of lines 44) or other features individually from a laser source at different orbital angles toward a surface 48, represented by multiple geometric shapes. The combined line images, recorded by a camera or other type of image sensor 66 from different angles but registered to geometric coordinates of the imaging system, provide structured light pattern information. Triangulation principles, using known distances such as base distance b between camera 66 and projector 64, are employed in order to interpret the projected light pattern and compute contour information for patient anatomy or other surface from the detected line deviation. Lines 44, or other projected pattern, can be visible light or light of infrared wavelengths not visible to the patient and to the viewer, but visible to the appropriate imaging sensors. An optional monitor 40 shows the acquired surface contour as reconstructed by computer processor logic using one or more surface contour reconstruction algorithms.

Other methods for obtaining the surface contour can alternately be used. Alternate methods include stereovision technique, structure from motion, and time-of-flight techniques, for example. The surface contour can be expressed as a mesh, using techniques familiar to those skilled in the contour imaging arts.

The surface acquisition system can use a structured light imaging technique, using one or more light sources and one or more light sensors as shown in FIG. 2B. The surface acquisition system projects, onto the patient, a known pattern of a light grid using the light sources. The deformed light pattern can be monitored by light sensors and analyzed by a host processor or computer to reconstruct a 3D surface model of the object. An exemplary structured light technique is described in Jason Geng, “Structured-light 3D surface imaging: a tutorial” Advances in Optics and Photonics, 2011. 3(2): p. 128-160, incorporated herein in its entirety by reference. Advantageously, 3D surface contour generation using structured light requires very little time for image acquisition and processing.

Both surface contour characterization and volume image content are used for motion compensation and correction of the present disclosure. This image content can be acquired from previously stored data that can be from the same imaging apparatus or from different apparatus. However, there can be significant advantages in obtaining the surface contour characterization and volume image content from the same apparatus, particularly for simplifying the registration task.

### Exemplary Apparatus

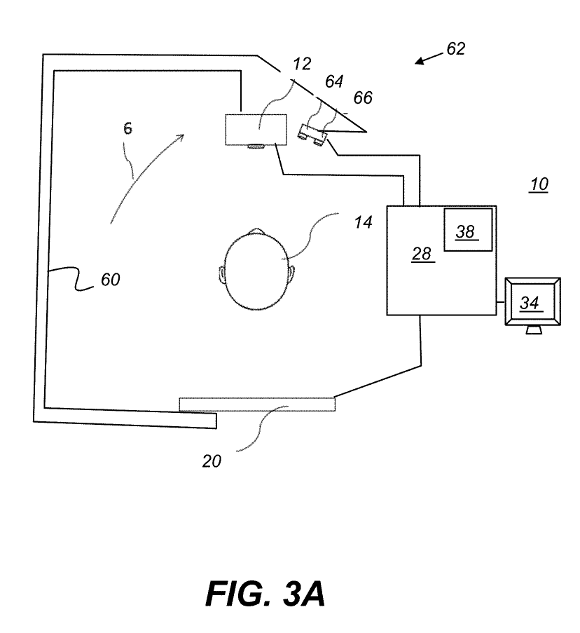

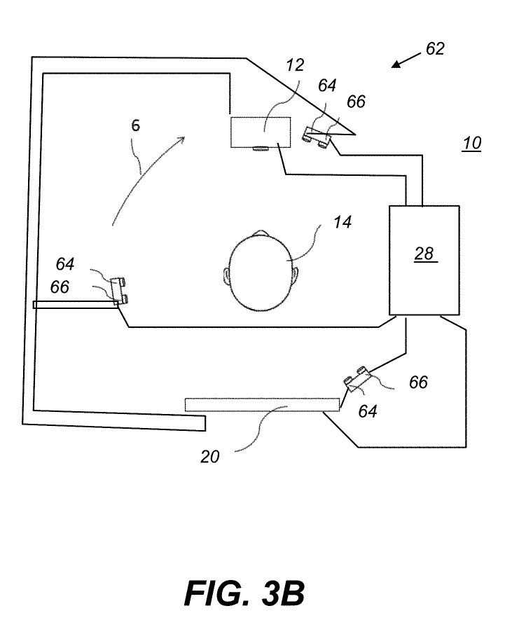

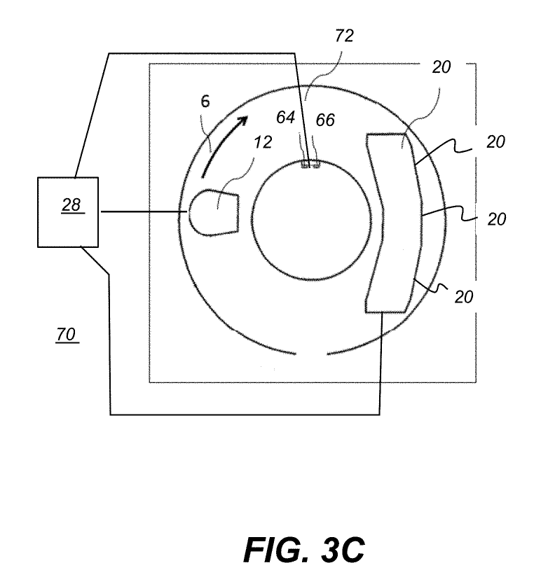

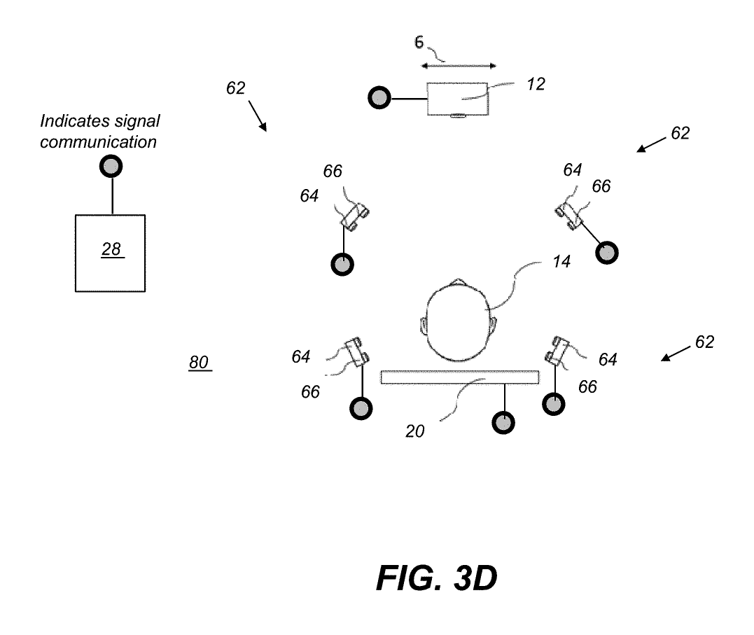

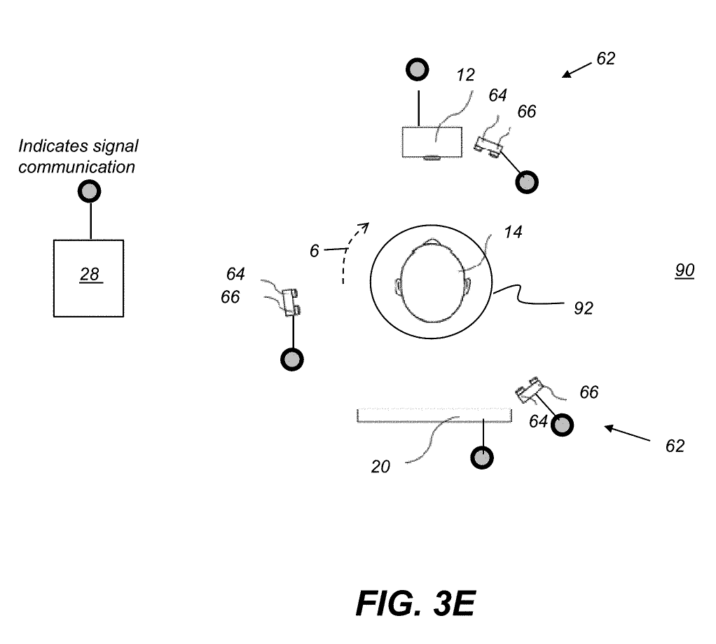

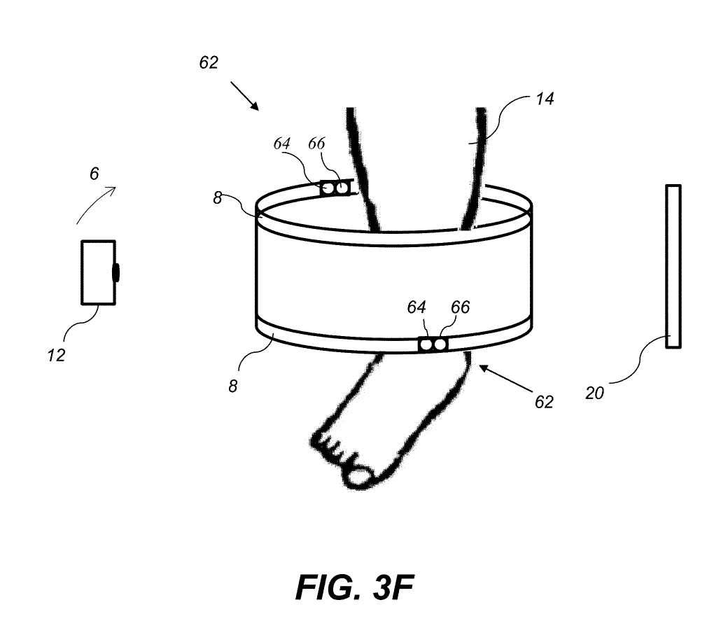

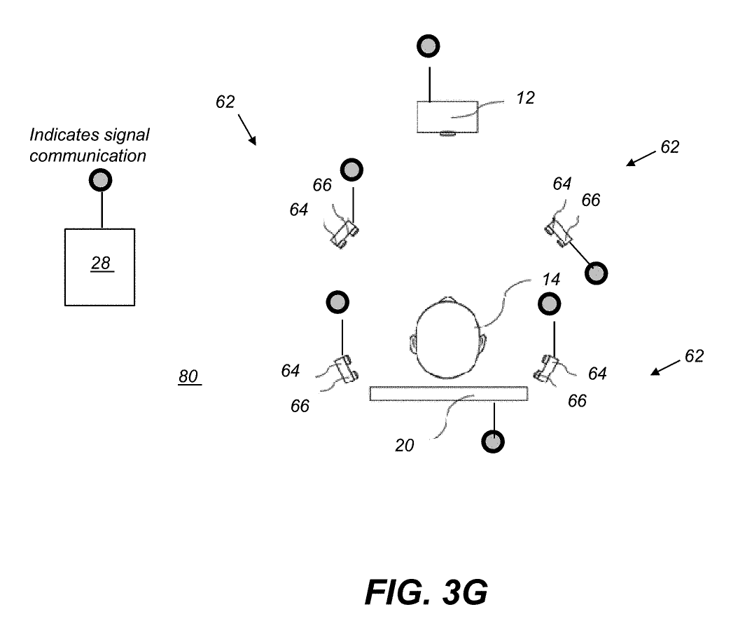

FIGS. 3A-3E and 3G show top view component configurations for a number of different imaging apparatus 10 configurations for acquiring both surface contour and reconstructed volume image data according to embodiments of the present disclosure. FIG. 3A shows an arrangement using a rotational gantry 60 that provides a transport apparatus for orbiting x-ray source 12 and detector 20 about subject 14, along with light scanner 62 for surface contour characterization having light pattern projector 64 and camera or sensor 66. A rotation direction 6 is shown. A control logic processor 28 is in signal communication with x-ray source 12, detector 20, and scanner 62 components for surface characterization. Control logic processor 28 shown in FIGS. 3A-3E can include a controller 38 that coordinates image acquisition between scanner 62 and the radiography apparatus in order to identify and characterize patient motion for control of image acquisition and to support subsequent processing of the x-ray projection image data. Control logic processor 28 can also include the logic for projection image processing and for volume CT image reconstruction as well as surface contour characterization, or may provide connection with one or more additional computers or processors that perform the volume or surface contour reconstruction function and display of volume imaging results, such as on display 34. The FIG. 3A configuration may serve, for example, for a dental imaging device using CBCT combined with structured light imaging.

FIG. 3B shows an arrangement with gantry 60 having x-ray source 12 and detector 20 and a number of pattern projectors 64 and cameras or sensors 66 that provide light scanner 62 for surface characterization.

FIG. 3C is a schematic diagram showing an MDCT (Multiple-Detector Computed Tomography) apparatus 70 that provides a single x-ray source 12 and a bank of multiple x-ray detectors 20 within a stationary bore 72. A projector 64 and camera 66 are also provided for contour imaging.

FIG. 3D is a schematic top view showing an imaging apparatus 80 for chest tomosynthesis having multiple pairs of light projectors 64 and sensors 66 as scanner 62, external to the x-ray acquisition components.

FIG. 3E is a schematic top view diagram that shows a computed tomography (CT) imaging apparatus 90 with stationary source 12 and detector 20 and rotating subject 14 on a support 92 that provides a transport apparatus for patient rotation. Stationary scanners 62 for surface contour acquisition are positioned outside the x-ray scanner hardware.

FIG. 3F is a schematic view diagram that shows an extremity X-ray imaging apparatus for volume imaging, having an x-ray source 12 and detector 20 configured to orbit about subject 14, and having multiple surface contour acquisition devices, scanners 62 that can move independently on rails 8 during projection data acquisition.

FIG. 3G is a schematic top view showing imaging apparatus 80 for chest radiographic imaging using multiple scanners 62 to provide multiple surface contour acquisition devices positioned outside of the imaging system.

The moving trajectories of the X-ray sources and X-ray detectors can be, for example, helix (e.g., MDCT), full circle (e.g., dental CBCT CT), incomplete circle (e.g., extremity CBCT), line, sinusoidal, and stationary (e.g., low-cost CT), or other suitable movement pattern.

### Motion Artifact Reduction

To help reduce motion artifacts in X-ray images, the Applicants propose a motion artifact reduction (MAR) system and method. The motion artifact reduction (MAR) system includes: a surface acquisition or characterization system for generating 3D surface models of a patient; an X-ray volume imaging apparatus for acquiring X-ray projection data of a patient; a controller to synchronize the surface acquisition system and the X-ray imaging apparatus; and a control logic processor (for example, a processor or other computing device that executes a motion reduction algorithm, or the like) that uses the X-ray projection data and 3D surface models to reconstruct a 3D volume, wherein the reconstructed volume has reduced patient motion artifacts.

In some cases, patient motion from a given position can be significant and may not be correctable. This can occur, for example, when the patient coughs or makes some other sudden or irregular movement. In the later reconstruction phase, the control logic processor 28 or controller 38 can suspend acquisition by the X-ray imaging system until the patient can recover the previous position.

The control logic can also analyze the acquired 3D surface image of the patient in real time and perform motion gating acquisition (also termed respiration gating) based on this analysis. With motion gating, surface contour acquisition can be associated with x-ray projection image acquisition and may even be used to momentarily prevent or defer acquisition. Using the controller to monitor and coordinate image acquisition, at least one 3D surface model of the patient can be obtained for each 2D X-ray projection. The acquired 3D surface model can be used for motion reduction in the reconstruction phase.

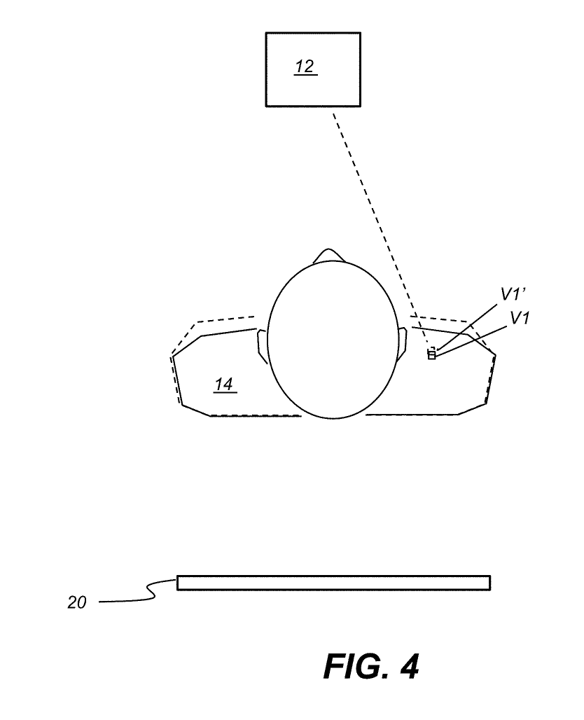

The schematic diagram of FIG. 4 shows one problem that is addressed for reducing motion artifacts according to an embodiment of the present disclosure. Normal patient breathing or other regular movement pattern can effectively change the position of a voxel V1 of subject 14 relative to x-ray source 12 and detector 20. At exhalation, the position of voxel V1 appears as shown. At full inhalation, the position shifts to voxel V1′. Without some type of motion compensation for each projection image, the wrong voxel position can be updated in 3D reconstruction.

Embodiments of the present disclosure provide motion compensation methods that characterize patient motion using imaging techniques such as surface contour imaging. A 3D surface model is generated from the acquired surface contour images and is used to generate transformation parameters that modify the volume reconstruction that is formed. Some exemplary transformation parameters include translation, rotation, skew, or other values related to feature visualization. Synchronization of the timing of surface contour imaging data capture with each acquired 2D x-ray projection image allows the correct voxel to be updated where movement has been detected. Because 3D surface contour imaging can be executed at high speeds, it is possible to generate a separate 3D surface contour image corresponding to each projection image 20 (FIG. 2A). Alternately, contour image data can be continuously updated, so that each projection image 20 corresponds to an updated 3D surface model.

There are two classic computational approaches used for 3D volume image reconstruction: (i) an analytic approach that offers a direct mathematical solution to the reconstruction process; and (ii) an iterative approach that models the imaging process and uses a process of successive approximation to reduce error according to a cost function or other type of objective function. Each of these approaches has inherent strengths and weaknesses for generating accurate 3D reconstructions.

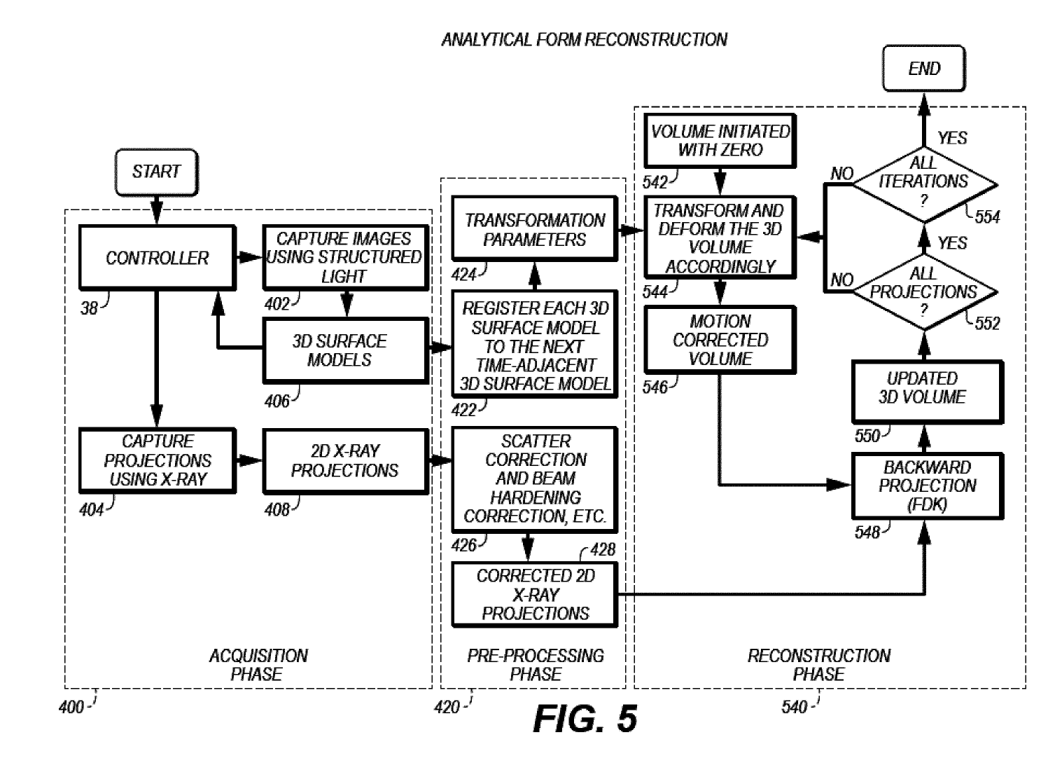

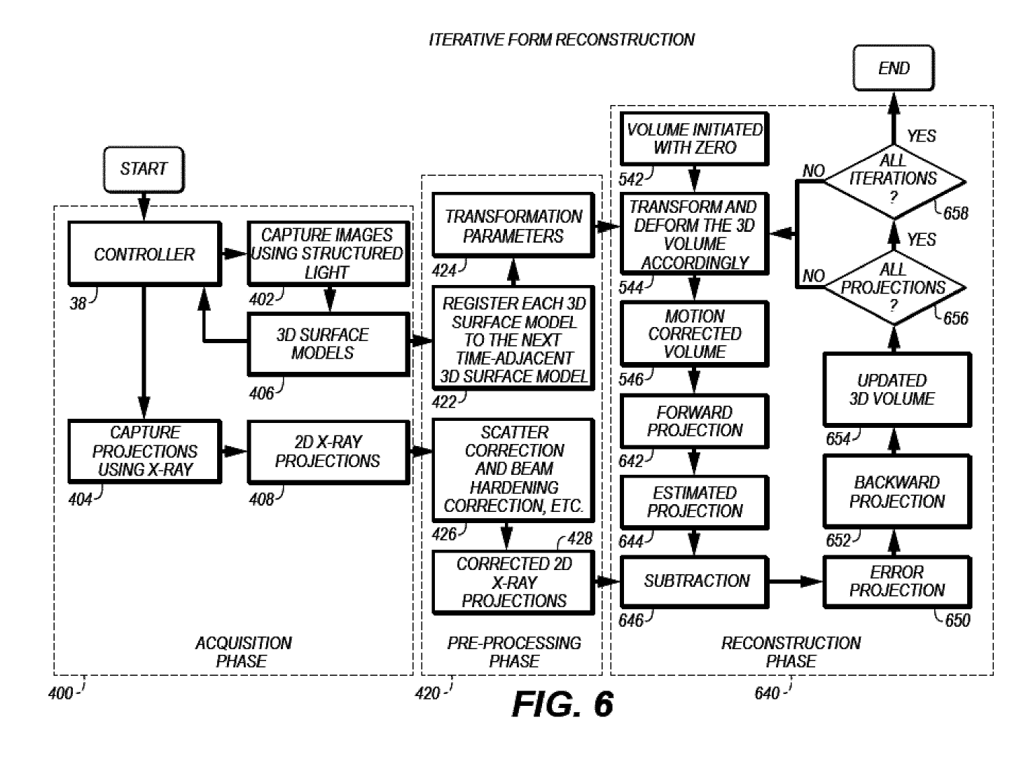

The logic flow diagram of FIG. 5 shows an overall process for integrating motion correction into volume data generation processing when using analytical techniques for volume reconstruction. Alternately, the logic flow diagram of FIG. 6 shows processing when using iterative reconstruction approaches for volume reconstruction. In both FIGS. 5 and 6, three phases are shown. The first two phases, an acquisition phase 400 and a pre-processing phase 420 are common whether analytic or iterative reconstruction is used. Following these phases, a reconstruction phase 540 executes for analytical reconstruction techniques or, alternately, a reconstruction phase 640 executes for iterative reconstruction techniques.

Referring to FIGS. 5 and 6, in acquisition phase 400, controller 38 captures 3D surface contour images, such as structured light images from scanner 62, in a scanning step 402. Controller 38 also coordinates acquisition of x-ray projection images 408 from detector 20 in a projection image capture step 404. Controller 38 and its associated control logic processor 28 use the captured 3D surface contour images to generate one or more 3D surface models in a surface model generation step 406 in order to characterize the surface contour at successive times, for synchronization of projection image data with surface contour information.

In pre-processing phase 420 of FIGS. 5 and 6, the 3D surface models generated from contour imaging can be registered to previously acquired surface models in a registration step 422. A set of transformation parameters 424 is generated for the surface contour data, based on changes detected in surface position from registration step 422. This transformation information uses the sequence of contour images and is generated based on changes between adjacent contour images and time-adjacent 3D surface models. 3D surface registration can provide and use rigid-object registration algorithms, such as to account for patient body translation and rotation, for example. In addition, 3D surface registration can provide and use deformable registration algorithms, such as to account for chest movement due to breathing and joint movement.

A correction step 426 then serves to provide a set of corrected 2D x-ray projections 428 for reconstruction. Correction step 426 can provide a number of functions, including scatter correction, lag correction to compensate for residual signal energy retained by the detector from previous images, beam hardening correction, and metal artifact reduction, for example.

Continuing with the FIG. 5 process, a reconstruction phase 540 using analytic computation then integrates surface contour imaging and x-ray projection imaging results for generating and updating a 3D volume 550 with motion compensation. Transformation parameters 424 from pre-processing phase 420 are input to a transformation step 544. Step 544 takes an initialized volume from an initiation step 542 and applies transformation parameters 424 from pre-processing phase 420 to generate a motion-corrected volume 546. The corrected 2D x-ray projections 428 and the motion-corrected volume data then go to a reconstruction step 548. Reconstruction step 548 executes backward projection, using the FDK (Feldkamp-Davis-Kress) algorithm as in the example shown in FIG. 5 or other suitable analytical reconstruction technique, to update the motion corrected volume 546. A decision step 552 determines whether or not all projection images have been processed. A decision step 554 then determines whether or not all iterations for reconstruction have been performed. At the completion of this processing, the reconstructed 3D volume is corrected for motion detected from surface contour imaging.

The FIG. 6 process uses iterative processing in its reconstruction phase 640. Transformation parameters 424 from pre-processing phase 420 are input to transformation step 544. Step 544 takes an initialized volume from initiation step 542 and applies transformation parameters 424 to generate motion-corrected volume 546. The iterative process then begins with a forward projection step 642 that performs forward projection through the 3D volume to yield an estimated 2D projection image 644. A subtraction step 646 then computes a difference between the estimated 2D X-ray projections and the corrected 2D X-ray projection to yield an error projection 650. The error projection 650 is used in a backward projection step 652 to generate an updated 3D volume 654 using a SART (simultaneous algebraic reconstruction technique) algorithm, statistical reconstruction algorithm, total variation reconstruction algorithm, or iterative FDK algorithm, for example. A decision step 656 determines whether or not all projection images have been processed. A decision step 658 then determines whether or not all iterations for reconstruction have been performed. Iterative processing incrementally updates the 3D volume until a predetermined number of cycles have been executed or until an error value between estimated and corrected projections is below a given threshold. At the completion of this processing, the reconstructed 3D volume is corrected for motion detected from surface contour imaging.

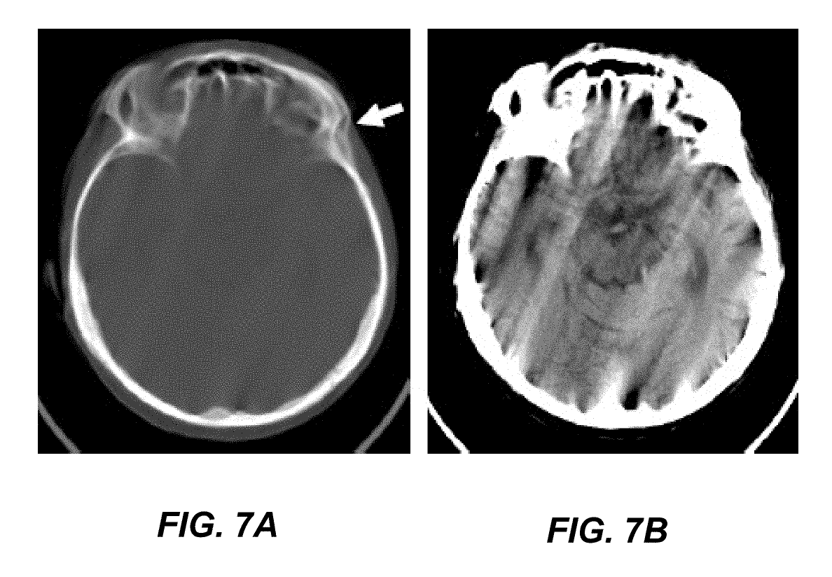

By way of example, images illustrating motion artifacts can be found in Boas, F. Edward, and Dominik Fleischmann. “CT artifacts: causes and reduction techniques. “Imaging in Medicine 4.2 (2012): 229-240.”, incorporated herein in its entirety. Motion artifacts can include blurring and double images, as shown in FIG. 7A and streaks across the image as shown in FIG. 7B.

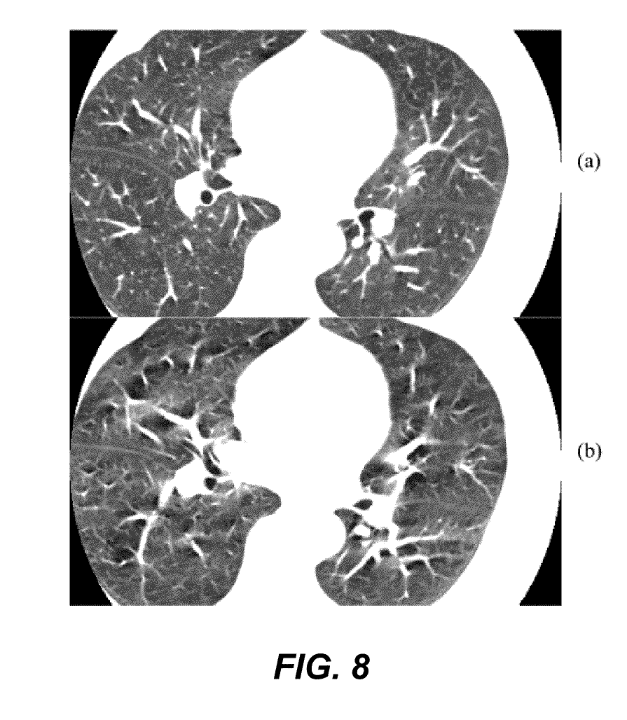

Reference is made to Hsieh, Jiang. “Computed tomography: principles, design, artifacts, and recent advances.” Bellingham, Wash.: SPIE, 2009, pages 258-269 of Chapter 7. This reference describes a respiratory motion artifact, as best illustrated in FIG. 8, wherein (a) is a chest scan relatively free of respiratory motion and (b), for the same patient, shows artifacts due to being scanned during breathing.

Accordingly, Applicants have disclosed a system for constructing a 3D volume of an object, comprising: a surface acquisition system for acquiring 3D surface images of the object; an X-ray imaging system for acquiring a plurality of X-ray projection images of the object; a controller to synchronize control the surface acquisition system and the X-ray imaging system to acquire the 3D surface images and X-ray projection images; and a processor to construct a 3D volume using the acquired 3D surface images and X-ray projection images.

Accordingly, Applicants have disclosed a method for reconstructing a 3D volume, comprising: providing a synchronized system comprised of a surface acquisition system and a X-ray imaging system; using the synchronized system, acquiring a plurality of surface images and a plurality of X-ray projection images of a patient; generating a plurality of 3D surface models of the patient using the plurality of surface images; and reconstructing the 3D volume using the plurality of X-ray projection images and the plurality of 3D surface models. In one embodiment, the step of reconstructing the 3D volume employs an analytical form reconstruction algorithm. In another embodiment, the step of reconstructing the 3D volume employs an iterative form reconstruction algorithm.

It can be appreciated that other processing sequences can alternately be executed using the combined contour image and projection image data to compensate for patient motion as described herein.

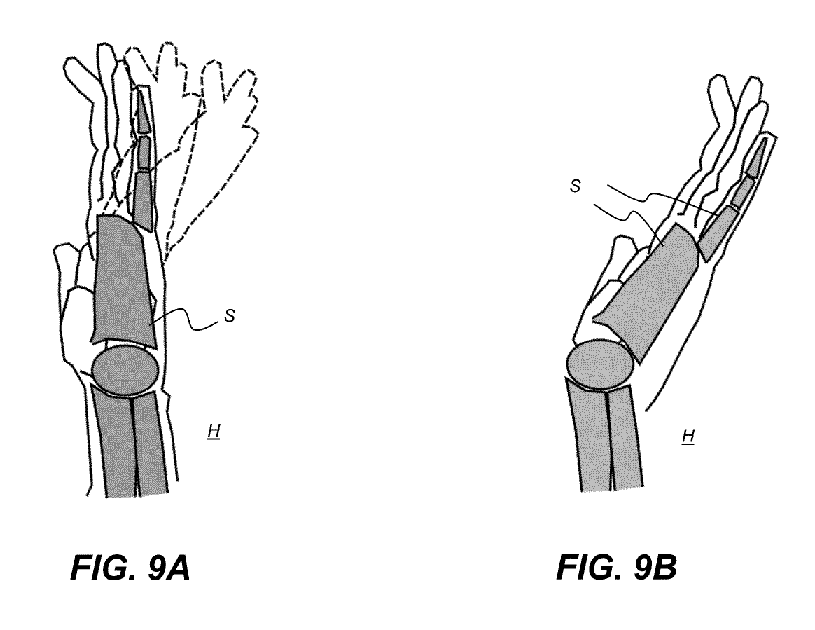

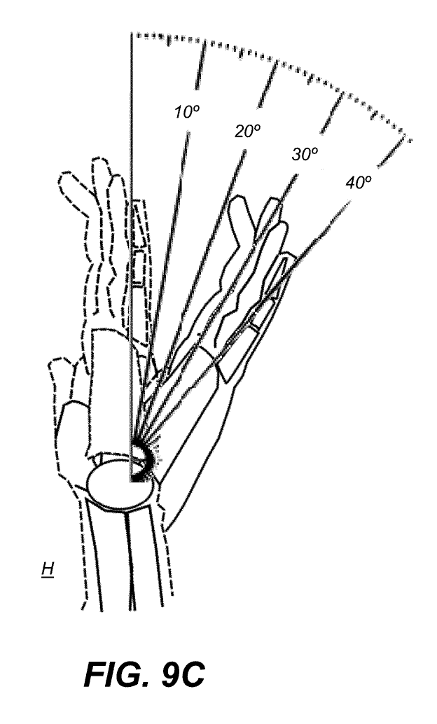

An embodiment of the present disclosure enables the various types of imaging apparatus 10, 70, 80, 90 shown in FIGS. 3A-3G to reconstruct 4D images of subject anatomy that show joint movement associated with time as the fourth dimension. 4D characterization of the changes to an image volume can be shown by a process that deforms obtained 3D image content according to detected surface movement. Referring to the schematic diagram of FIGS. 9A, 9B, and 9C, there are shown positions of a hand bending or hand extension during a volume imaging exam that records patient movement. Image content acquired as part of the exam is represented for both the surface of the anatomy and the skeletal portions, with a portion of the bone structures S schematically represented in shaded form in FIGS. 9A and 9B. Radiographic projection images, such as from a CBCT apparatus as shown in FIGS. 3A and 3B, can be acquired at the angular positions shown in FIGS. 9A and 9B, as well as at any number of intermediate positions. Patterned illumination images for 3D surface imaging are also acquired at the FIG. 9A and 9B positions, as well as at many more angular positions of the hand intermediate to the FIG. 9A and 9B positions. Radiographic images from different angular positions can also be obtained during the motion sequence. For this example, FIG. 9C shows the full arc of movement between the positions of FIG. 9A and 9B at approximately 30 degrees relative to the ulna or radius at the wrist.

By acquiring radiographic image data and surface contour image data throughout the movement sequence shown in FIG. 9A and FIG. 9B, embodiments of the present method can obtain sufficient information for interpolating volume image content for the intermediate positions. With respect to the coarse reference measurements overlaid onto FIG. 9C, interpolation methods of the present disclosure are able to generate additional intermediate volume images between the beginning and ending 0 and 30 degree positions, such as at 10 and 20 degree positions, for example. These angular values are given by way of example and not limitation; resolution with respect to angular and translational movement can be varied over a broad range, depending on the requirements of the imaging exam.

A few possible intermediate positions are represented in dashed outline in FIG. 9A. In the context of the present disclosure, “index positions” are movement positions or poses in the movement series at which are obtained one or more radiographic projection images (such as from a CBCT volume imaging apparatus) and a surface contour image (such as an image obtained using structured illumination with the apparatus of FIG. 2B). The volume image generated for an index position can be termed an “index volume image” in the context of the present disclosure.

Using a combination of surface characterization and radiographic projection images obtained at and between index positions allows analysis of joint movement without requiring the significant number of exposures that would otherwise be required to reconstruct full 3D radiographic volume data for each of numerous intermediate positions in a movement sequence such as that shown in FIGS. 9A-9C.

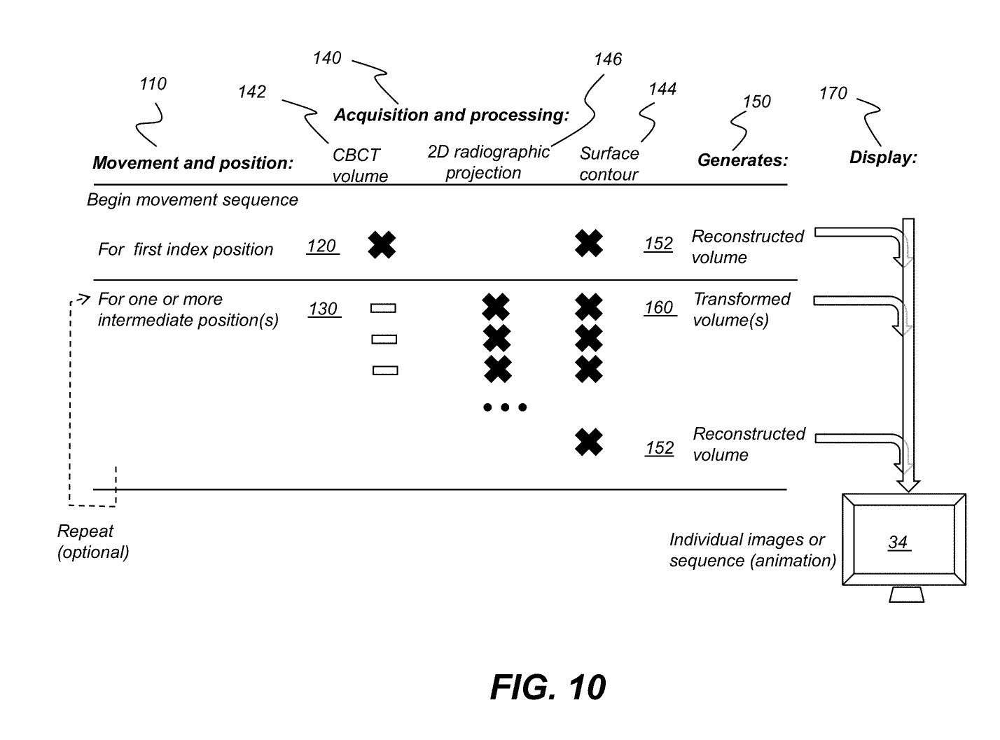

The logic flow diagram of FIG. 10 shows a sequence of steps that can be used for 4D volume imaging according to an embodiment. Under guidance from a technician or practitioner, a movement sequence 110 is executed by the patient for the examination. During movement sequence 110, an ongoing acquisition and processing step 140 executes. A first position can be an index position 120 as described previously. In the embodiment shown in FIG. 10, both a 3D image volume 142, such as an image reconstructed from a series of CBCT radiographic image projections, and a surface contour image or characterization 144 are obtained at the first index position 120. Acquiring the full index volume image is optional; alternately, radiographic image data acquired at successive stages during the movement sequence can be used for generating the full volume. A volume generation step 150 generates a combined reconstructed volume 152 for index position 120.

In the FIG. 10 process, two types of images are acquired at subsequent positions in the movement sequence. 2D radiographic projections 146 of the moving subject are acquired at different angles as the patient moves. During this movement, a number of surface contour images are also obtained to provide surface contour characterization 144. Using iterative reconstruction processing, one or more transformed volumes 160 are generated by a sequence that:
(i) forms a volume using the 3D surface contour data;
(ii) transforms the position and orientation of skeletal features in conformance with the volume construction; and
(iii) corrects the volume transformation according to an error value obtained by comparing a forward projection at an angle through the reconstructed volume with an actual radiographic 2D projection at the same angle.

In iterative processing, the cycle of steps (ii) and (iii) repeats any number of times until the calculated error obtained in (iii) is within acceptable limits or is negligible. The result of this processing is a transformed or reconstructed volume.

It can be appreciated that the basic procedure shown in FIG. 10 can allow for a number of modifications. For example, the index position 120 is optional; generation of the volume reconstruction can begin with surface contour characterization acquired throughout the movement sequence and radiographic projections progressively acquired and incrementally improved as the movement sequence proceeds. A full CBCT volume can optionally be acquired and processed at any point in the movement sequence as an index volume; this can be helpful to validate the accuracy of the ongoing transformation results.

After acquisition and processing of images for volume image reconstruction, a display step 170 then executes. Display step 170 can display some portion or all of the movement sequence on display 34, with the transformed volumes 160 generated for each movement position displayed in an ordered sequence. As shown in FIG. 10, the acquisition and processing sequence can repeat throughout the movement sequence 110 until terminated, such as by an explicit operator instruction.

A number of different reconstruction tools can be used for generating the reconstructed volume 152 or transformed volumes 160 of FIG. 10. Exemplary reconstruction methods known to those skilled in the volume imaging arts include both analytic and iterative methods, such as simultaneous algebraic reconstruction technique (SART) algorithms, statistical reconstruction algorithms, total variation reconstruction algorithms, and iterative FDK (Feldkamp Davis Kress) reconstruction algorithms.

The processing task for generating the transformed volume image can apply any of a number of tools, including using at least one of rigid transformation, non-rigid transformation, 3D-to-3D transformation, surface-based transformation, 3D-to-2D registration, feature-based registration, projection-based registration, and appearance-based transformation, for example.

At each of a succession of time-adjacent intermediate positions, the sequence of FIG. 10 obtains both surface contour characterization 144 and 2D radiographic projections 146. The surface contour characterization 144 can be used to iteratively transform the volume model that is currently in use, in order to update the transformed volume 160 so that it is appropriate for each successive corresponding intermediate position 130. In addition to transformation of the volume shape and boundaries, it is also desirable to obtain radiographic data throughout the motion sequence in order to check the accuracy of transformation for internal structures. This function can be performed by acquiring the 2D radiographic projection at the intermediate position 130 of anatomy motion. Slight errors in positioning of internal features between adjacent generated volumes can then be corrected using the acquired radiographic data.

Forward projection through the generated transformed volume helps to reconcile the existing volume deformation with acquired data for an intermediate position 130. A forward projection computed through the transformed volume image data generates a type of digitally reconstructed radiograph (DRR), a synthetic projection image that can be compared against the acquired radiographic projection image as part of iterative reconstruction. Discrepancies can help to correct for positioning error and verify that the movement sequence for a subject patient has been correctly characterized.

According to an alternate embodiment of the present disclosure, the following sequence can be used for updating the volume at each intermediate position 130:
a) generating a transformed volume image 160 for a specified intermediate position according to a surface contour characterization;
b) adjusting the generated transformed volume image 160 according to patient anatomical structures, implants, or other features;
c) comparing one or more forward projections of the adjusted transformed volume image with corresponding acquired 2D x-ray projection image(s); and
d) updating and displaying the transformed volume image 160 according to comparison results.

The update of the transformed volume image in d) above can use back projection algorithms, such as filtered back projection (FBP) or may use iterative reconstruction algorithms.

With particular respect to volume transformation methods and 2D-to-3D registration methods, reference is hereby made to the following, by way of example:
a) Yoshito Otake et al. “Robust 3D-2D image registration: application to spine interventions and vertebral labeling in the presence of anatomical deformation”, Physics in Medicine and Biology, (2013); 58(23): pp. 8535-53.
b) Piyush Kanti Bhunre et al. “Recovery of 3D Pose of Bones in Single 2D X-ray Images”, IEEE Applications of Computer Vision, 2007; pp. 1-6.
c) Taehyun Rhee et al. “Adaptive Non-rigid Registration of 3D Knee MRI in Different Pose Spaces” IEEE International Symposium on Biomedical Imaging: From Nano to Macro, 2008, pp. 1-4.
d) J. B. Mainz and Max A. Viergever, “A survey of medical image registration” in Medical Image Analysis, vol. 2, issue 1, March, 1998, pp. 1-36.
e) Zhiping Mu, “A Fast DRR Generation Scheme for 3D-2D Image Registration Based on the Block Projection Method”, IEEE Conference on Computer Vision and Pattern Recognition Workshops (CVPRW), 2016, pp. 169-177.

It can be appreciated that the sequence shown in FIG. 10 can be used to generate a motion picture sequence that can show a practitioner useful information about aspects of joint movement for a patient. Advantageously, embodiments of the present disclosure enable 4D image content to be generated and displayed without requiring the full radiation dose that would otherwise be required to capture each frame in a motion picture series.

It should also be noted that while the present disclosure describes the use of structured light imaging for surface contour characterization, other methods that employ reflectance imaging or other non-ionizing radiation could alternately be used for surface contour characterization.

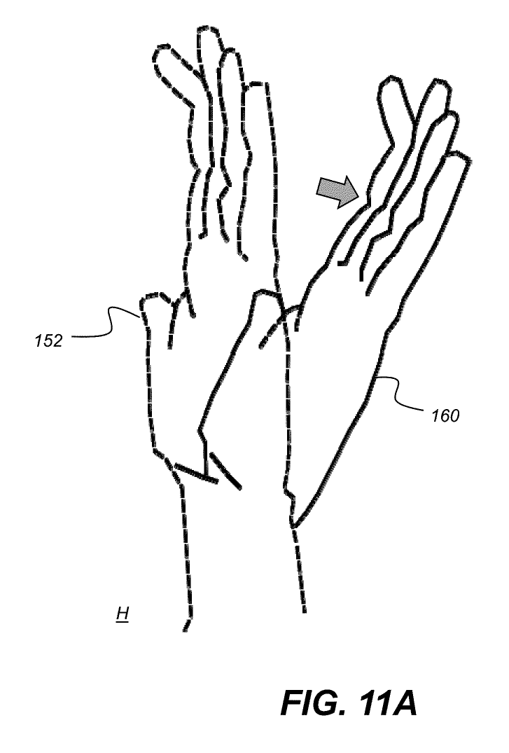

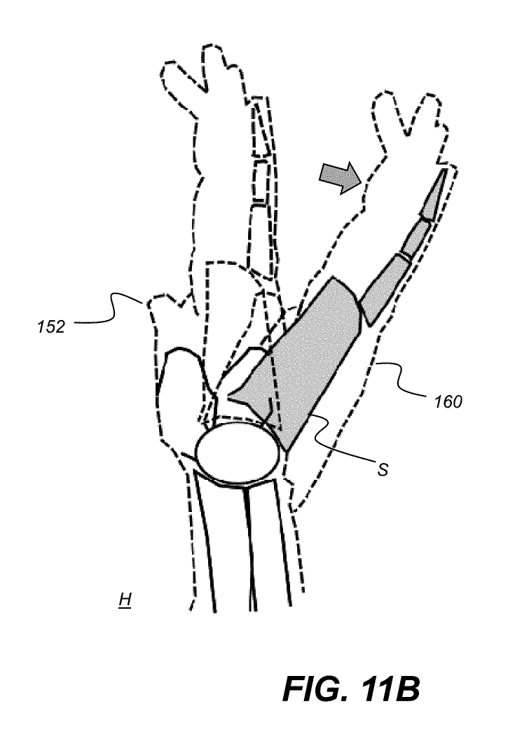

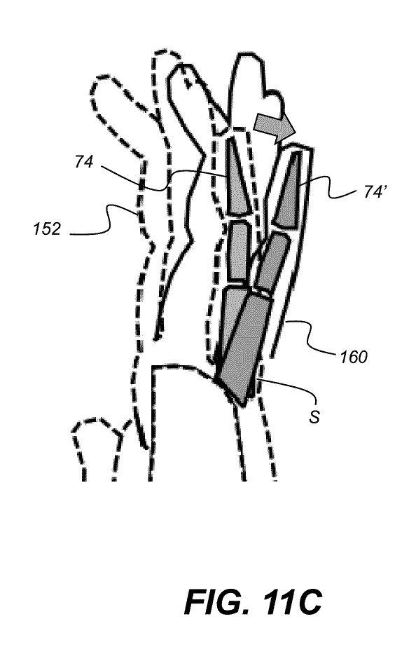

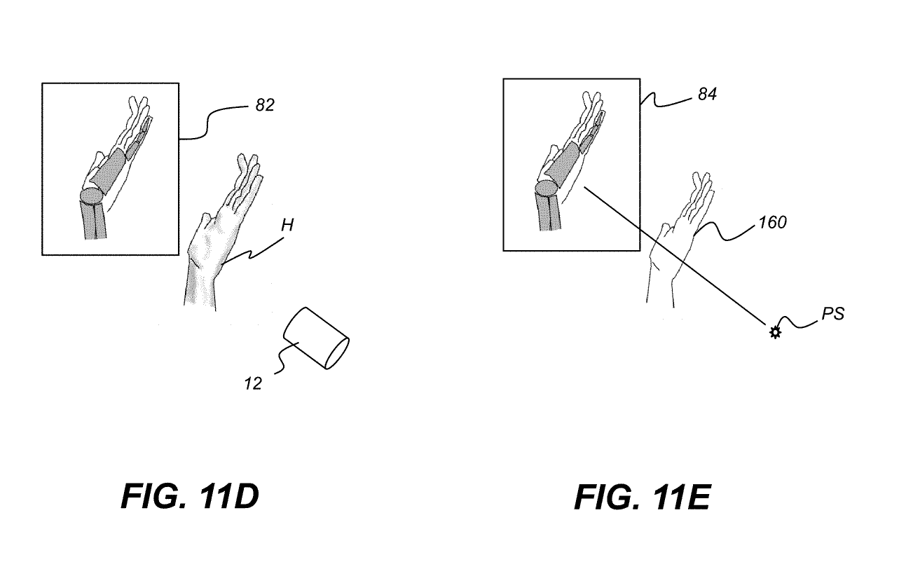

FIGS. 11A-11E show aspects of transformed volume 160 generation in schematic form and exaggerated for emphasis. FIG. 11A shows basic volume transformation for the reconstructed volume 152, according to 3D surface contour characterization. As shown, the surface contour information provides information that allows deformation of the original volume, with a corresponding change in position.

FIG. 11B and the enlarged view of FIG. 11C show how the 3D transformation is applied to the skeletal features and other inner structure of the imaged subject. FIG. 11B represents movement of inner bone structure S of hand H based initially on the 3D volume deformation.

FIG. 11C shows, in enlarged form, aspects of the operation performed in volume transformation used to generate transformed volume 160. A small portion of the volume image is represented for this description. Reconstructed volume 152 is represented in dashed lines; transformed volume 160 is represented in solid lines. According to the movement shown in changing to transformed volume 160, a distal phalange 74 is transformed in position to distal phalange 74′, with other skeletal structures also translated appropriately. Initial transformation is based on volume deformation, as described previously. Appropriate methods for translation and distortion of 3D features based on volume transformation are known to those skilled in the image manipulation arts.

According to an embodiment of the present disclosure, information for iterative reconstruction of the transformed image is available from comparison of a forward projection through the initially transformed volume with the actual radiographic projection image. This arrangement is shown in FIGS. 11D and 11E. FIG. 11D shows a radiographic projection image 82 acquired using an x-ray source 12 disposed at a particular angle with respect to the subject, hand H. FIG. 11E shows a synthetic forward projection 84. The image data that forms synthetic forward projection 84 is generated by calculating the projection image that would be needed for radiation from an idealized point source PS to contribute appropriate data content for forming transformed volume 160. Comparison of acquired radiographic image 82 with calculated forward projection image 84 then provides information that is useful for determining how closely the transformed volume 160 resembles the actual subject, hand H in the example shown, and indicating what adjustments are needed in order to more closely model the actual subject.

The corrected image projections can be used to help generate additional projection images for use in volume reconstruction, using methods well known to those skilled in the volume reconstruction arts.

Advantageously, the method of the present invention allows accurate 3D modeling of a motion sequence using fewer radiographic images than conventional methods, with lower radiation dose to the patient. By acquiring radiographic images in conjunction with 3D surface contour imaging content, the method allows the image subject to be accurately transformed with movement from one position to the next, with continuing verification and adjustment of calculated data using acquired image content.

Consistent with an embodiment, the present invention utilizes a computer program with stored instructions that control system functions for image acquisition and image data processing for image data that is stored and accessed from external devices or an electronic memory associated with acquisition devices and corresponding images. As can be appreciated by those skilled in the image processing arts, a computer program of an embodiment of the present invention can be utilized by a suitable, general-purpose computer system, such as a personal computer or workstation that acts as an image processor, when provided with a suitable software program so that the processor operates to acquire, process, transmit, store, and display data as described herein. Many other types of computer systems architectures can be used to execute the computer program of the present invention, including an arrangement of networked processors, for example.

The computer program for performing the method of the present invention may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing the method of the present invention may also be stored on computer readable storage medium that is connected to the image processor by way of the internet or other network or communication medium. Those skilled in the image data processing arts will further readily recognize that the equivalent of such a computer program product may also be constructed in hardware.

It is noted that the term “memory”, equivalent to “computer-accessible memory” in the context of the present disclosure, can refer to any type of temporary or more enduring data storage workspace used for storing and operating upon image data and accessible to a computer system, including a database. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer can also be considered to be a memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It is understood that the computer program product of the present invention may make use of various image manipulation algorithms and processes that are well known. It will be further understood that the computer program product embodiment of the present invention may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present invention, are not specifically shown or described herein and may be selected from such algorithms, systems, hardware, components and elements known in the art.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A system for reconstructing a 4D image, comprising:
a surface acquisition system for generating a 3D surface model of an object;
an X-ray imaging system for acquiring at least one 2D X-ray projection image of the object;
a controller to control the surface acquisition system and the X-ray imaging system; and
a processor to apply a 4D reconstruction algorithm/method to the 3D surface model and the at least one 2D X-ray projection to reconstruct a 4D X-ray volume of the imaged body part in motion.

2. The system of claim 1, wherein the surface acquisition system comprises:
one or more light sources adapted to project a known pattern of light grid onto the object;
one or more optical sensors adapted to capture a plurality of 2D digital images of the object; and
a surface reconstruction algorithm for reconstructing the 3D surface model of the object using the at least one 2D projection image.

3. The system of claim 2, wherein the light sources and the optical sensors are adapted to be either:
(i) mounted to a rotational gantry of the X-ray imaging system,
(ii) affixed to the bore of the X-ray imaging system, or
(iii) placed outside of (separate from) the X-ray imaging system.

4. The system of claim 1, wherein the X-ray imaging system comprises:
one or more X-ray sources adapted to controllably emit X-rays; and
one or more X-ray detectors including a plurality of X-ray sensors adapted to detect X-rays that are emitted from the X-ray sources and have traversed the object.

5. The system of claim 4, wherein the X-ray sources and X-ray detectors move in a trajectory, wherein the trajectory includes, but is not limited to, a helix, full circle, incomplete circle, line, sinusoid, and stationary.

6. The system of claim 1, wherein the controller synchronizes the surface imaging system and the X-ray imaging system.

7. The system of claim 1, wherein the 4D reconstruction algorithm/method comprises:
an X-ray projection correction process to generate a corrected 2D X-ray projection;
a 3D surface deformation process to deform each 3D surface model to the next time-adjacent 3D surface model and generate at least one transformation parameter;
a 3D volume deformation process to deform the volume under reconstruction according to the at least one transformation parameter;
a 3D volume deformation process to deform the volume under reconstruction according to the 2D X-ray projection using an anatomical structure or implant; and
an analytical form reconstruction process or an iterative form reconstruction process.

8. The system of 7, wherein the X-ray projection correction process includes a scatter correction, a beam hardening correction, or a metal artifact reduction correction.

9. The system of claim 7, further comprising a 3D surface registration algorithm comprising a rigid-object registration algorithm or a deformable registration algorithm.

10. The system of claim 7, wherein the analytical form reconstruction process includes an FDK (Feldkamp-Davis-Kress) algorithm.

11. The system of claim 7, wherein the iterative form reconstruction process includes a SART algorithm, a statistical reconstruction algorithm, a total variation reconstruction algorithm, or an iterative FDK algorithm.

12. The system of claim 7, wherein the 4D reconstruction method is applied until a predetermined threshold criterion is met.

13. A method comprising:
a) acquiring one or more radiographic images of patient anatomy at a first position;
b) acquiring a first surface contour image of the patient anatomy at the first position;
c) acquiring a second surface contour image of the patient anatomy after patient movement to a second position;
d) continuously acquiring additional radiographic images of the patient anatomy after patient movement from the first to the second position;
e) generating one or more transformed volume images of the patient anatomy according to the additionally acquired surface contour and radiographic images; and
f) displaying, storing, or transmitting one or more portions of the one or more transformed volume images.

14. The method of claim 13 wherein generating the one or more transformed volume images comprises comparing a computed forward projection image with an acquired radiographic image.

15. The method of claim 13 wherein acquiring the radiographic images comprises acquiring the images using a cone-beam computed tomography system.

16. The method of claim 13 wherein acquiring the first surface contour image comprises acquiring at least one structured light image.

17. The method of claim 17 wherein displaying at least the transformed volume comprises displaying a motion picture image series showing portions of the generated transformed volume images.

18. The method of claim 13 wherein generating the transformed volume image comprises using at least one of rigid transformation, non-rigid transformation, 3D-to-3D transformation, surface-based transformation, 3D-to-2D registration, feature-based registration, projection-based registration, and appearance-based transformation.

19. The method of claim 13 wherein generating the one or more transformed volume images comprises using a reconstruction algorithm taken from the list consisting of a simultaneous algebraic reconstruction technique algorithm, a statistical reconstruction algorithm, a total variation reconstruction algorithm, and an iterative FDK algorithm.

20. A method comprising:
a) acquiring one or more radiographic images of patient anatomy at a first position;
b) acquiring a first surface contour image of the patient anatomy at the first position;
c) acquiring a second surface contour image of the patient anatomy during patient movement to a second position;
d) continuously acquiring additional radiographic images of the patient anatomy during patient movement from the first to the second position;
e) generating a volume image of the patient and one or more transformed volume images of the patient anatomy according to the acquired surface contour and radiographic images; and
f) displaying, storing, or transmitting portions of the one or more transformed volume images.

21. The method of claim 20 further comprising acquiring a volume image of patient anatomy at a first position in the movement sequence.

22. The method of claim 20 further comprising
(i) calculating a forward projection image of the transformed volume image at the second position;
(ii) comparing the calculated forward projection image with the acquired 2D radiographic projection at the second position; and
(iii) reconstructing the transformed volume image corresponding to the second position to form an updated volume image according to the comparison from step (ii).

23. The method of claim 22 wherein reconstructing uses an algorithm taken from the list comprising a simultaneous algebraic reconstruction technique algorithm, a statistical reconstruction algorithm, a total variation reconstruction algorithm, and an iterative FDK algorithm.
